(19) United States
(12) Patent Application Publication
(10) Pub. No.: US 2007/0038252 A1
(43) Pub. Date: Feb. 15, 2007

(54) Apparatus for surface electrical stimulation and stabilization to treat disorders of the joints

(76) Inventor: William J. Carroll, La Center, WA (US)

Correspondence Address: BLANK ROME LLP, 600 NEW HAMPSHIRE AVENUE, N.W., WASHINGTON, DC, 20037, US

(21) Appl. No.: 11/500,907

(22) Filed: Aug. 9, 2006

Related U.S. Application Data
(60) Provisional application No. 60/706,445, filed on Aug. 9, 2005.

(51) Int. Cl. A61N 1/00

(52) U.S. Cl. 607/2

(57) ABSTRACT

The present invention is an apparatus having a stabilizing device, at least two signal applicators, and a signal generator for surface electrical stimulation and stabilization to treat disorders of the joints.

### REFERENCES TO RELATED APPLICATIONS

The present application claims the benefit of U.S. Provisional Application No. 60/706,445, filed on Aug. 9, 2005, whose disclosure is hereby incorporated by reference in its entirety. The present application is also related to U.S. patent application Ser. No. 10/659,278, filed on Sep. 11, 2003.

### BACKGROUND OF THE INVENTION

1. Field of the Invention

The present invention relates to an apparatus for surface electrical stimulation and stabilization to treat disorders of the joints.

2. Description of the Related Art

Degenerative Joint Disease (DJD) and Osteoarthritis (OA) are disorders of the joints whose main symptoms are pain and restricted movement. DJD and OA typically strike the joints of the lower extremities, such as the hips, knees, ankles or toes, and hence, greatly decrease the freedom of mobility of a patient. The elderly population is the most susceptible to DJD and OA.

Joints consist of bones and soft tissue structures that are designed to move and tolerate the activities of daily living. Each joint is encapsulated in a protective sac-like structure called a bursa. The lining of the joint, called the synovium, produces synovial fluid that bathes and lubricates the articular surfaces of the joint and helps protect the cartilage, a rubbery tissue that guards the bone.

DJD and OA are progressive disease processes. The breakdown of cartilage that is seen with these conditions occurs in several steps. First, the synovial fluid becomes thinner and loses its elasticity and viscosity, which decreases its ability to cushion the knee joint. Without this cushioning effect, the cartilage in the joint may be more likely to “wear down,” and begins to deteriorate. Next, the surface of the smooth cartilage covering the joint softens and it begins to lose its ability to absorb the impact of movement and can be more easily damaged from excess use or shock. Finally, the joint may lose its shape as the cartilage breaks down and bony growths or bone spurs may form on the edges of the effected joint compartment. Small particles of bone or cartilage may float around in the joint space. All of these occurrences cause pain in the affected joint and greatly restrict its natural movement.

A typical standard of care for treatment of DJD and OA is Nonsteroidal anti-inflammatory drugs (NSAIDs). The efficacy of NSAIDs to treat DJD and OA may be dependent upon their analgesic or anti-inflammatory properties, or the slowing of degenerative processes in the cartilage. NSAIDs are commonly known to have toxic effects in the stomach, gastrointestinal tract, liver and kidney.

Another typical standard of care for treatment of DJD and OA is bracing of the joint. Typical braces relieve pain by reducing the compressive forces on the joint being braced. Braces may be worn for long periods of time and are especially effective at reducing pain from DJD and OA when the wearer is physically active. Numerous braces are described in the art, examples of which are described below.

U.S. Pat. No. 5,458,565 to Tillinghast, III describes an osteoarthritic knee brace having flexible upper and lower arm members rotatably connected to each other by a rotary hinge assembly and an inflatable or deflatable fluid-containing pad positioned between the hinge assembly and the knee joint. The upper and lower arm members are stressed away from the knee joint, thereby applying a restoring force to the knee joint across the hinge assembly and the pad.

U.S. Published Patent Application No. 2006/0135900 to Ingimundarson et al describes a knee brace having a flexible proximal shell and a flexible distal shell. The proximal and distal shells each include a body portion and a compliant edge. The compliant edges are formed from flexible resilient material that is configured to extend beyond the outer perimeter of the body portion of each the proximal and distal shells.

Another typical standard of care for DJD and OA is surface electrical stimulation (SES). SES increases blood flow to stimulated areas and has a beneficial effect on the production and quality of the synovium and the resultant synovial fluid. SES provides an effective, non-surgical, non-pharmaceutical method of treating DJD and OA that patients can self-administer.

U.S. Pat. No. 5,273,033 to Hoffman describes a method for treating arthritic joint symptoms by applying electrical impulses via electrodes. The electrical impulses are initially applied with an amplitude sufficiently high to be sensed by the patient, followed by continuing application at a reduced amplitude subsensory to the patient. This method allows for a reduction of pain and joint stiffness, while helping to increase the range of motion of the joint.

U.S. patent application Ser. No. 10/659,278, referenced above, describes a method and device for improving the production and quality of synovial fluid in a joint through SES. Use of the method and device described causes a decrease in the progression of joint deterioration.

U.S. Pat. Nos. 6,393,328 and 6,988,005 to McGraw et al. describe portable electro-medical devices that are easy to use and have safety features that make them amenable to home use. The devices described are capable of applying many types of electro-medical treatment, including various types of stimulation that can be used for SES, as described below.

While the above methods of treating OA and DJD have been used with varying levels of effectiveness, there remains a need in the art for methods and devices that allow patients greater mobility with relief of OA and DJD symptoms, while simultaneously allowing the symptoms to be treated. A combination brace with SES can have greater benefit than stimulation alone or bracing alone. The present invention is used for the treatment and amelioration of joint disorders of, but not limited to, the hips, knees, ankles, toes, back, neck and shoulders. The present invention is used to treat a variety of joint disorders no matter how caused, such as through degenerative processes, wear and tear, or other causes. The present invention is used to prevent the development of a joint disorder, wherein the joint disorder is predicted due to factors including, but not limited to, generic predisposition or prior injury to a joint.

### SUMMARY OF THE INVENTION

The present invention addresses the above-described conditions and needs, and an object of the present invention is to provide an apparatus for surface electrical stimulation and stabilization to treat disorders of the joints.

According to one aspect of the present invention, there is provided an apparatus for imparting an electrical signal into an articular segment of the human body. The apparatus has a stabilizer that actively supports the articular segment while allowing for substantial mobility, at least two signal applicators mounted within the stabilizer, and a signal generator electrically connected to the signal applicators for transmitting electrical stimulation to the articular segment.

In one aspect of the present invention, the stabilizer causes the relief of pain by alleviating pressure on at least one compartment of the joint while the signal applicators deliver electrical stimulation for treatment of the joint. As the stabilizer allows for substantial mobility, the wearer is free to move about and be physically active while wearing the apparatus.

In a specific example of the present invention, there is provided an apparatus for imparting an electrical signal into a leg and knee portion of the human body. The apparatus has a knee brace for stabilizing the leg and knee portion while allowing for substantial mobility, at least two signal applicators mounted within the knee brace and a signal generator electrically connected to the signal applicators for transmitting electrical stimulation to the leg and knee portion.

In another aspect of the present invention, the apparatus imparts an electric signal that mimics a pattern of normal physical activity by sequencing at least two muscle groups.

In a still further aspect of the present invention, the apparatus is designed for convenient home use. A patient wishing to treat a joint disorder may simply wear the apparatus while going about their daily activities. Further, as the signal applicators may be fixed in place within the stabilizer, they may be positioned by a medical professional so that the signal applicators will be in the proper spatial orientation relative to the joint to be treated each time the apparatus is worn. That way, proper signal applicator positioning may be assured and the instances of incorrect signal applicator placement by the patient may be greatly reduced.

The above and further objects and features of the invention will more fully be apparent from the following detailed description with accompanying drawings.

### BRIEF DESCRIPTION OF THE DRAWINGS

FIG. 1 is a perspective, front-side view of an apparatus for surface electrical stimulation and stabilization to treat disorders of the joints according to one embodiment of the present invention, showing a stabilizer, a pair of signal applicators, and a signal generator;

FIG. 2 is a perspective view showing a right-side corresponding to FIG. 1;

FIG. 3 is a perspective view showing a back-side corresponding to FIGS. 1 and 2;

FIG. 4 is a perspective view showing a left-side corresponding to FIGS. 1, 2 and 3;

FIG. 5 is a perspective, front-side view of the apparatus for surface electrical stimulation and stabilization, featuring a double-hinged knee brace;

FIG. 6 is a perspective view showing a front-side of an apparatus for surface electrical stimulation and stabilization to treat disorders of the joints according to one embodiment of the present invention, showing signal applicator placement for bathing the knee joint with electrical stimulation;

FIG. 7 is a perspective, right-side view corresponding to FIG. 6; and

FIG. 8 is a perspective view showing a left-side corresponding to FIGS. 6 and 7.

### DETAILED DESCRIPTION OF THE INVENTION

Referring to the drawings, wherein like reference numerals represent like parts throughout the various drawing figures, reference numeral 10 is directed to an apparatus for surface electrical stimulation and stabilization to treat disorders of the joints. The apparatus 10 has a stabilizer for stabilizing a joint or articular segment of the human body, a signal generator 30 and at least two signal applicators 50, 60.

In the embodiments set forth below, the articular segment described is a leg L and the joint being treated is a knee K. However, it is also contemplated that the present invention may be used for electrically stimulating and stabilizing other articular segments with joints including, but not limited to, elbows, ankles, shoulders, hips, toe joints and finger joints.

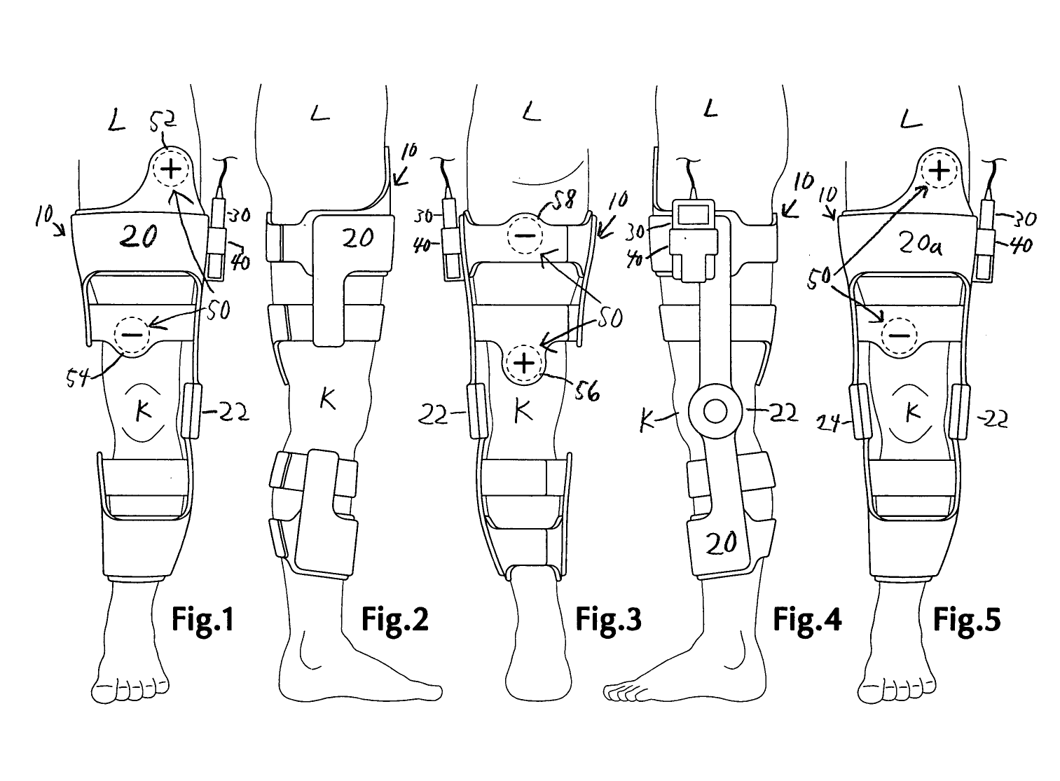

In a preferred embodiment, and with reference to FIGS. 1-4, the stabilizer is typically a brace 20 that is designed to reduce the compressive forces on at least one compartment of the knee K. Typically, the medial compartment of the knee K is usually effected more than the lateral compartment. A single-hinge 22 brace 20, as shown in FIGS. 1-4 can be used to provide a lateral thrust to oppose the pathologically unbalanced load within the medial compartment of the knee K. Additionally, the brace 20 may remove weight bearing stress from the arthritic or otherwise affected compartment and transfer it onto the healthy compartment. The reduction of compressive forces on the knee K compartments results in less pain and wear of the joint. The brace 20 is usually worn for long periods of time, especially when the patient is physically active.

Alternatively, a double-hinge 22, 24 brace 20a can be used, as illustrated in FIG. 5.

In addition to the unloader-type brace 20 embodiment shown in FIGS. 1-5, it is also contemplated that apparatus 10 may be configured with alternative orthoses, such as stabilizers, compression units, slings, dynamic splints, range-of-motion splints, and the like that assist in the movement of the spine and limbs.

The signal generator 30 is a stimulator, such as the portable electro-medical device described by McGraw et al. in U.S. Pat. Nos. 6,393,328 and 6,988,005. The signal generator 30 of the apparatus 10 includes controls for controlling the amplitude and frequency of the electrical signals, at least one output cable, and a power source.

Referring to FIGS. 1-5, the signal generator 30 can be attached directly to the brace 20 via a holder or fastener 40. In another embodiment of the invention as shown in FIG. 7, the electro-medical device 30 can be detachable from the brace 20 while remaining electrically connected to the signal applicators 50, 60 via the output cable.

The signal generator 30 is electrically connected to the signal applicators 50, 60 via the output cable of the signal generator 30 to produce electrical stimulation for the passing of various types of current across the skin. Examples of this type of stimulation include, but are not limited to, Transcutaneous Electrical Nerve Stimulation (TENS), NeuroMuscular Electrical Stimulation (NMES), Interferential Stimulation, Diadynamic Stimulation, High Volt Galvanic Stimulation (HVGS), Electro-Magnetic and Pulsed Electro-Magnetic Field Stimulation (EMF & PEMF) and Micro-current Stimulation.

In another embodiment, apparatus 10 can be configured for use with other stimulators and electro-medical devices that allow for generating an electrical signal to be applied to the articular segment of the body.

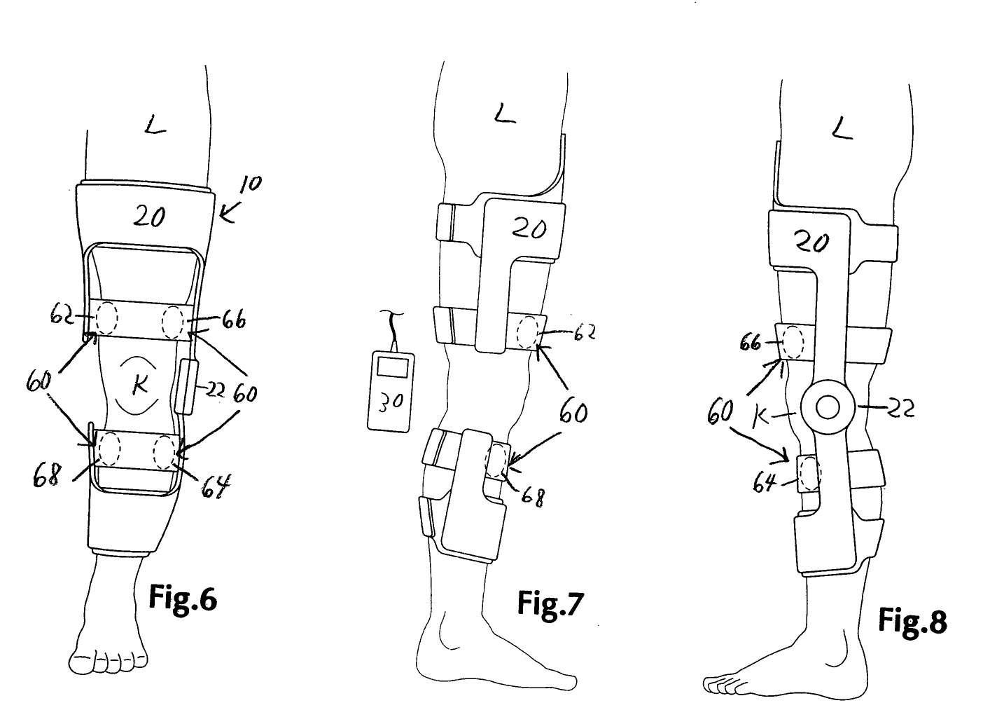

Referring to FIGS. 1-4 and to FIGS. 6-8, the signal applicators 50, 60 of the present invention 10 are mounted in the brace 20. In a preferred embodiment of the invention 10, the signal applicators 50, 60 may be electrode pads 52, 54, 56, 58 attached to the inner surface or skin-side surface of the brace 20. The electrode pads 52, 54, 56, 58 are double-sided or double-stick pads for adhesively adhering to the inner surface of the brace 20 and the skin of the joint L. The electrode pads 52, 54, 56, 58 are reusable, disposable and replaceable and can be removed from the brace 20 without disrupting the connectivity with the hard-wiring and leads array from the pads 52, 54, 56, 58 to the output cable of the signal generator 30. The electrode pads 52, 54, 56, 58 are also repositionable within the brace 20 with the use of conventionally known straps that secure the brace 20 to the leg L. It should be apparent to one of skill in the art that any plurality of electrode pads and electrode pads of varying sizes may be used to provide a therapeutic pattern of therapy.

The apparatus 10 stimulates the effector muscle groups to simulate a pattern of normal action and function of the joint and its surrounding muscles that mimics activities of daily living (walking, running), but does not produce the destructive wear and tear on the knee K that would normally be seen in a weight bearing situation. As show in FIGS. 1 and 3, the electrode pad 52 is placed at an anterior, proximal, lateral portion on the vastus lateralis of the leg L. The electrode pad 54 is placed at an anterior, distal medial portion of the vastus medialis obliquus of the leg L. The electrode pad 56 is placed at a posterior, distal, paramedian plane portion of the biceps femoris of the leg L, and the electrode pad 58 is placed at a posterior, proximal, paramedian plane portion of the biceps femoris of the leg L. The pads 52, 54, 56, 58 are repositionable within the sized-to-fit brace 20 to achieve the desired effector muscle group placement.

With further reference to the electrode pads 52, 54, 56, 58, the first electrode pad 52 is preferably an anode and the second electrode pad 54 is a preferably a cathode electrically connected to the signal generator 30 on a first channel. The third electrode pad 56 is preferably a second anode and the fourth electrode pad 58 is preferably a second cathode both electrically connected to the signal generator 30 on a second channel. The amplitude for this sequential or pattern type stimulation that mimicks the normal action of the affected joint can range from about 5 mA to about 100 mA as rated into a 500 ohm load, and the duration of stimulation can range from about 10 minutes to about 10 hours per day. This stimulation produces the normal vegetative functions of the knee K, such as production of synovial fluid without further degradation of the knee K.

Another embodiment of the present invention is shown in FIGS. 6-8. In FIGS. 6-8, the signal applicators 60 are electrode pads 62, 64, 66, 68 that may be placed to surround the knee K, both proximally and distally, to bathe the area with electrical stimulation. The pads 62, 64, 66, 68 are repositionable with the sized-to-fit brace 20 and other orthoses via the use of conventional straps and fasteners commonly used to secure the brace 20 to the leg L.

With continuing reference to the electrode pads 62, 64, 66, 68, the first pair of electrode pads 62, 64 and the second pair of electrode pads 66, 68 may form a criss-cross pattern to box-in the knee K, as shown in FIG. 6. A first pair of electrode pads 62, 64 may be adapted to form a first circuit on a first channel of the signal generator 30 having a first frequency. A second pair of electrode pads 66, 68 may be adapted to form a second circuit on a second channel of the signal generator 30 having a second frequency. The two frequencies share an interference relationship with one another to produce at least one beat frequency. With this type of continuous, interferential stimulation transmitted via the paired electrode pads 62, 64 and 66, 68 the amplitude can range from about 0.1 mA to about 100 mA as rated into a 500 ohm load, and the duration of stimulation can range from about 10 minutes to about 10 hours per day.

Alternative stimulation patterns, including those that do not simulate natural function, are also contemplated for use in the present invention 10.

Although the invention has been disclosed in its preferred forms, the specific embodiments thereof as disclosed and illustrated herein are not to be considered in a limiting sense, because numerous variations are possible. The subject matter of the invention includes all novel and non-obvious combinations and sub-combinations of the various elements, features, functions, and/or properties disclosed herein. The following claims define certain combinations and sub-combinations of features, functions, elements, and/or properties that are regarded as novel and non-obvious. Other combinations and sub-combinations may be claimed through amendment of the present claims or presentation of new claims in this or a related application. Such claims, whether they are broader, narrower, equal, or different in scope to any earlier claims, also are regarded as included within the subject matter of the invention.

What is claimed is:

1. An apparatus for imparting an electrical signal into an articular segment of the human body, comprising:
a means for stabilizing the articular segment, wherein said means for stabilizing actively supports the articular segment for substantial mobility;
at least two signal applicators mounted to said means for stabilizing for applying electrical stimulation to said articular segment; and,
a signal generator electrically connected to said signal applicators for supplying them with electrical signals for stimulating the articular segment.

2. The apparatus of claim 1, wherein said means for stabilizing is a brace that envelopes a knee joint of the human body and provides medial and lateral thrusts to oppose at least one pathologically unbalanced load within at least one compartment of the knee joint.

3. The apparatus of claim 1, wherein said at least two signal applicators are located on the articular segment such that the electrical stimulation mimics a pattern of normal physical activity by sequencing at least two muscle groups.

4. The apparatus of claim 2, wherein said at least two signal applicators are located on the articular segment such that the electrical stimulation mimics a pattern of normal physical activity by sequencing at least two muscle groups.

5. The apparatus of claim 3, wherein said at least two signal applicators are comprised of first, second, third and fourth electrode pads for adhering to the surface of the skin of the human body, said electrode pads having an electrically conductive relationship between the electrical signals and the skin.

6. The apparatus of claim 5, wherein said first electrode pad is an anode and said second electrode pad is a cathode electrically connected to a first channel of said signal generator.

7. The apparatus of claim 6, wherein said third electrode pad is a second anode and said fourth electrode pad is a second cathode electrically connected to a second channel of said signal generator.

8. The apparatus of claim 1, wherein said at least two signal applicators are two pairs of electrode pads connected to said signal generator so that a first pair of electrode pads are adapted to form a first circuit on a first channel having a first frequency and a second pair of electrode pads are adapted to form a second circuit on a second channel having a second frequency, said second frequency having an interference relationship with said first frequency to produce at least one beat frequency.

9. The apparatus of claim 8, wherein said two pairs of electrode pads are positioned to surround the articular segment both proximally and distally.

10. The apparatus of claim 8, wherein the articular segment is bathed in the electrical signals.

11. The apparatus of claim 1, wherein said signal generator comprises:
a means for controlling the amplitude of the electrical signal;
a means for controlling the frequency of the electrical signal;
at least one output cable; and,
a power source.

12. The apparatus of claim I, wherein the electrical signals create at least one of Transcutaneous Electrical Nerve Stimulation, NeuroMuscular Electrical Stimulation, Interferential Stimulation, Diadynamic Stimulation, High Volt Galvanic Stimulation, Electro-Magnetic and Pulsed Electro-Magnetic Field Stimulation and Micro-current Stimulation.

13. The apparatus of claim 1, wherein said means for stabilizing is a range-of-motion orthosis adapted to apply torque to the articular segment.

14. The apparatus of claim 1, wherein the signal applicators are removably mounted to the means for stabilizing.

15. The apparatus of claim 1, wherein the signal applicators may be mounted in varying positions within the means for stabilizing.

16. An apparatus for imparting an electrical signal into an articular segment of the human body, comprising:
a stabilizer for stabilizing the articular segment, wherein the stabilizer actively supports the articular segment for substantial mobility;
at least two signal applicators mounted to the stabilizer for applying electrical stimulation to said articular segment; and,
a signal generator electrically connected to said signal applicators for supplying them with electrical signals for stimulating the articular segment.

17. The apparatus of claim 16, wherein the stabilizer is a brace that envelopes a knee joint of the human body and provides medial and lateral thrusts to oppose at least one pathologically unbalanced load within at least one compartment of the knee joint.

18. The apparatus of claim 16, wherein said at least two signal applicators are located on the articular segment such that the electrical stimulation mimics a pattern of normal physical activity by sequencing at least two muscle groups.

19. The apparatus of claim 16, wherein said at least two signal applicators are comprised of first, second, third and fourth electrode pads for adhering to the surface of the skin of the human body, said electrode pads having an electrically conductive relationship between the electrical signals and the skin.

20. The apparatus of claim 19, wherein said first electrode pad is an anode and said second electrode pad is a cathode electrically connected to a first channel of said signal generator.

21. The apparatus of claim 20, wherein said third electrode pad is a second anode and said fourth electrode pad is a second cathode electrically connected to a second channel of said signal generator.

22. The apparatus of claim 16, wherein said at least two signal applicators are two pairs of electrode pads connected to said signal generator so that a first pair of electrode pads are adapted to form a first circuit on a first channel having a first frequency and a second pair of electrode pads are adapted to form a second circuit on a second channel having a second frequency, said second frequency having an interference relationship with said first frequency to produce at least one beat frequency.

23. The apparatus of claim 22, wherein said two pairs of electrode pads are positioned to surround the articular segment both proximally and distally.

24. The apparatus of claim 22, wherein the articular segment is bathed in the electrical signals.

25. The apparatus of claim 16, wherein said signal generator comprises:
a means for controlling the amplitude of the electrical signal;
a means for controlling the frequency of the electrical signal;
at least one output cable; and,
a power source.

26. The apparatus of claim 16, wherein the electrical signals create at least one of Transcutaneous Electrical Nerve Stimulation, NeuroMuscular Electrical Stimulation, Interferential Stimulation, Diadynamic Stimulation, High Volt Galvanic Stimulation, Electro-Magnetic and Pulsed Electro-Magnetic Field Stimulation and Micro-current Stimulation.

27. The apparatus of claim 16, wherein the stabilizer is a range-of-motion orthosis adapted to apply torque to the articular segment.

28. The apparatus of claim 16, wherein the signal applicators are removably mounted to the stabilizer.

29. The apparatus of claim 16, wherein the signal applicators may be mounted in varying positions within the stabilizer.

30. An apparatus for imparting an electrical signal into a leg and a knee portion of the human body, comprising:
a knee brace for stabilizing the leg and the knee portion, wherein said brace actively supports the leg and the knee portion for substantial mobility;
two or more electrode pads mounted within said knee brace, wherein said electrode pads are adapted to adhere to the surface of the skin, said electrode
pads are associated with an electrically conductive relationship between the electrical signal and the skin; and,
a stimulator electrically connected to said electrode pads for providing electrical stimulation to at least one of the knee and the leg.

31. The apparatus of claim 30, wherein the electrical stimulation mimics a pattern of normal physical activity by sequencing at least two muscle groups, and said two or more electrode pads further comprising:
a first electrode pad for placement at an anterior, proximal, lateral portion on the vastus lateralis of the leg;
a second electrode pad for placement at an anterior, distal medial portion of the vastus medialis obliquus of the leg;
a third electrode pad for placement at a posterior, distal, paramedian plane portion of the biceps femoris of the leg; and,
a fourth electrode pad for placement at a posterior, proximal, paramedian plane portion of the biceps femoris of the leg.

32. The apparatus of claim 31, wherein said first electrode pad is an anode and said second electrode pad is a cathode electrically connected to said stimulator on a first channel.

33. The apparatus of claim 31, wherein said third electrode pad is a second anode and said fourth electrode pad is a second cathode electrically connected to said stimulator on a second channel.

34. The apparatus of claim 30, wherein said two or more electrode pads are two pairs of electrode pads connected to said stimulator so that a first pair of electrode pads are adapted to form a first circuit on a first channel having a first frequency and a second pair of electrode pads are adapted to form a second circuit on a second channel having a second frequency, said second frequency having an interference relationship with said first frequency to produce at least one beat frequency.

35. The apparatus of claim 30, wherein the electrical signal electrical signals create at least one of Transcutaneous Electrical Nerve Stimulation, NeuroMuscular Electrical Stimulation, Interferential Stimulation, Diadynamic Stimulation, High Volt Galvanic Stimulation, Electro-Magnetic and Pulsed Electro-Magnetic Field Stimulation and Micro-current Stimulation.

36. The apparatus of claim 30, wherein the signal applicators are removably mounted within the knee brace.

37. The apparatus of claim 30, wherein the signal applicators may be mounted in varying positions within the knee brace.
